(19) United States
(12) Patent Application Publication
(10) Pub. No.: US 2015/0282996 A1
(43) Pub. Date: Oct. 8, 2015

(54) Male Medical Undergarment and Method

(71) Applicants: Anthony James Newton, Chicago, IL (US); Kobie Jawanza Newton, Chicago, IL (US); Jimmie Mae Newton, Chicago, IL (US)

(72) Inventors: Anthony James Newton, Chicago, IL (US); Kobie Jawanza Newton, Chicago, IL (US); Jimmie Mae Newton, Chicago, IL (US)

(21) Appl. No.: 13/854,051

(22) Filed: Mar. 30, 2013

(51) Int. Cl. A61F 13/491

(52) U.S. Cl. CPC A61F 13/4915

(57) ABSTRACT

A medical undergarment comprises a waistband configured to be worn on a waist of a man, a front panel connectable to a front region of the waistband and comprising an inner opening, an adjustable strap connecting the front panel to a rear region of the waistband, an absorbent cup comprising an absorbent material, receivable in the inner opening of the front panel, and configured to contain and absorb urine, and a fastener configured to removably connect the front panel to the front region of the waistband.

### BACKGROUND OF THE INVENTION

The present invention relates to a male medical undergarment and method.

Presently, a solution for urine collection includes a horizontal pad with tape to stick to undergarments. When moving, this horizontal pad compresses, causing leakage to one's underwear. In some cases, the solution is in the form of under shorts, like women's padded underwear. For the male, activity is restricted for fear of leakage because the horizontal shaped pad compresses and the garment sticks to the skin and leaks when one is active. With the horizontal pad and the panty-padded underwear there is leakage to one's underwear or entire undergarment and outer clothes areas.

As can be seen, there is a need for solutions to these and other problems.

### SUMMARY OF THE INVENTION

In one aspect of the present invention, a medical undergarment comprises: a waistband configured to be worn on a waist of a man; a front panel connectable to a front region of the waistband and comprising an inner opening; an adjustable strap connecting the front panel to a rear region of the waistband; an absorbent cup comprising an absorbent material, receivable in the inner opening of the front panel, and configured to contain and absorb urine; and a fastener configured to removably connect the front panel to the front region of the waistband.

In one aspect, the front panel further comprises an adjustment device configured to hold at least one of a penis and a scrotum in place. In one aspect, the adjustment device comprises an elastic band. In one aspect, the adjustment device comprises a fabric mesh. In one aspect, the fastener comprises a hook-and-loop fastener. In one aspect, the absorbent pad has an approximately semi-ellipsoid shape.

In one aspect, the medical undergarment further comprises a length adjustor connected to the adjustable strap and configured to allow the adjustable strap to be adjusted in length.

In one aspect, a method of collecting urine comprises: providing the medical undergarment as described; inserting the absorbent cup into the inner opening; placing a penis of a user into the absorbent cup; adjusting the adjustable strap; and connecting the front panel to the front region of the waistband with the fastener.

In one aspect, the front panel further comprises an adjustment device configured to hold the penis in place, and wherein the method further comprises adjusting the adjusting device so as to hold the penis in place. In one aspect, the adjustment device wraps tightly around the penis so as to prevent outflow of urine from the inner opening.

These and other features, aspects and advantages of the present invention will become better understood with reference to the following drawings, description and claims.

### BRIEF DESCRIPTION OF THE DRAWINGS

FIG. 1: is a perspective view of one embodiment of the present invention.

FIG. 2: is an exploded perspective view of one embodiment of the present invention.

FIG. 3: is a side elevation view of one embodiment of the present invention.

FIG. 4: is a cross-sectional view of one embodiment of the present invention taken on line 4-4 of FIG. 1.

FIG. 5: is an exploded cross-sectional view of one embodiment of the present invention.

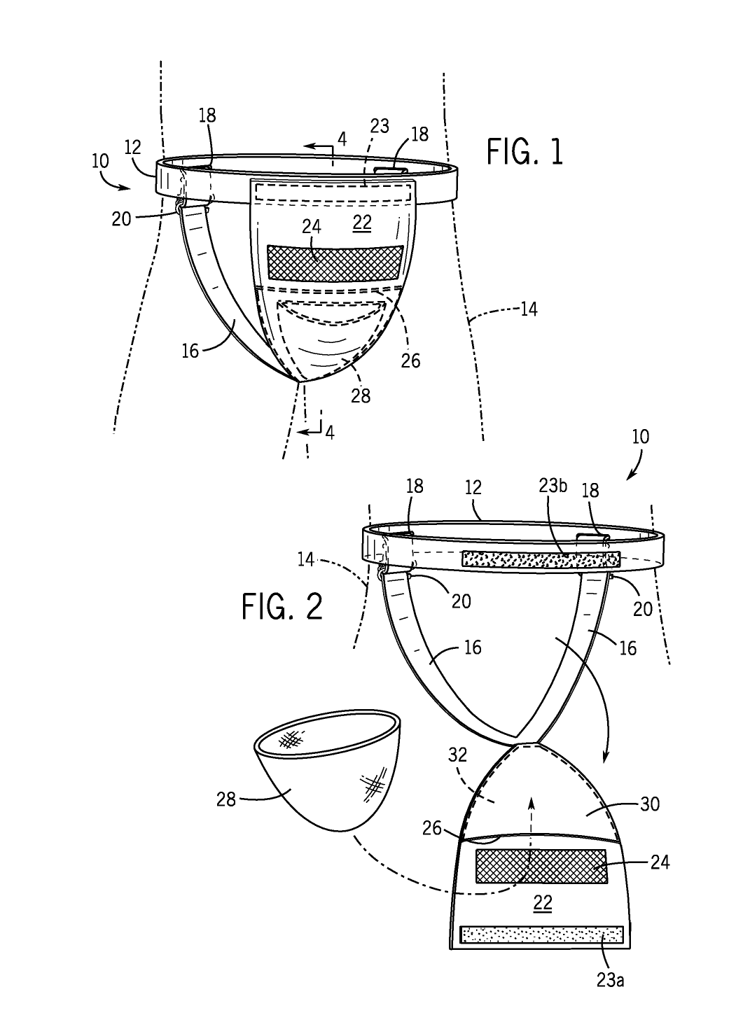

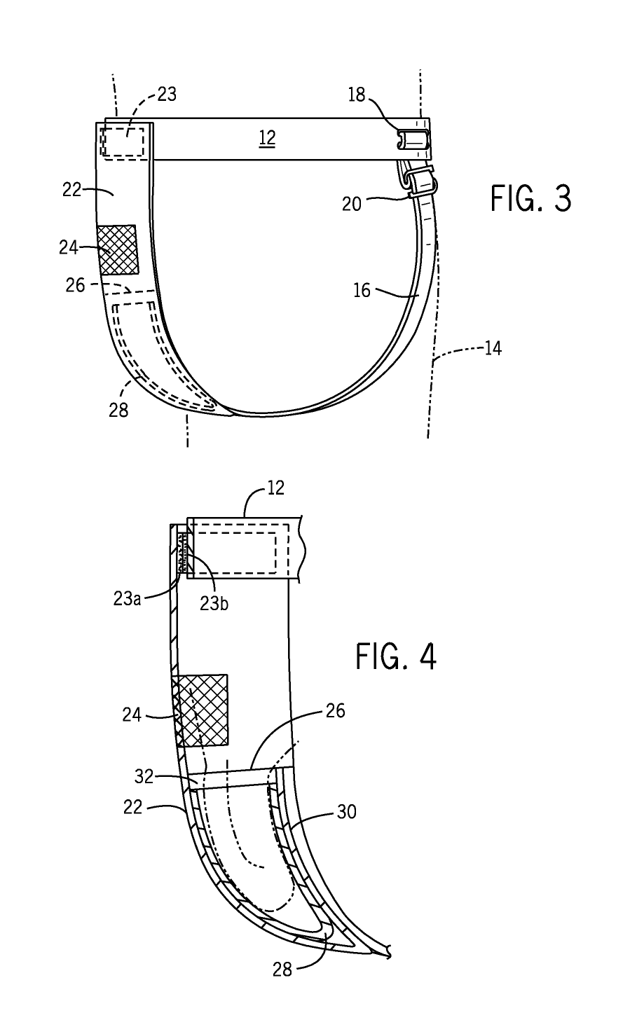

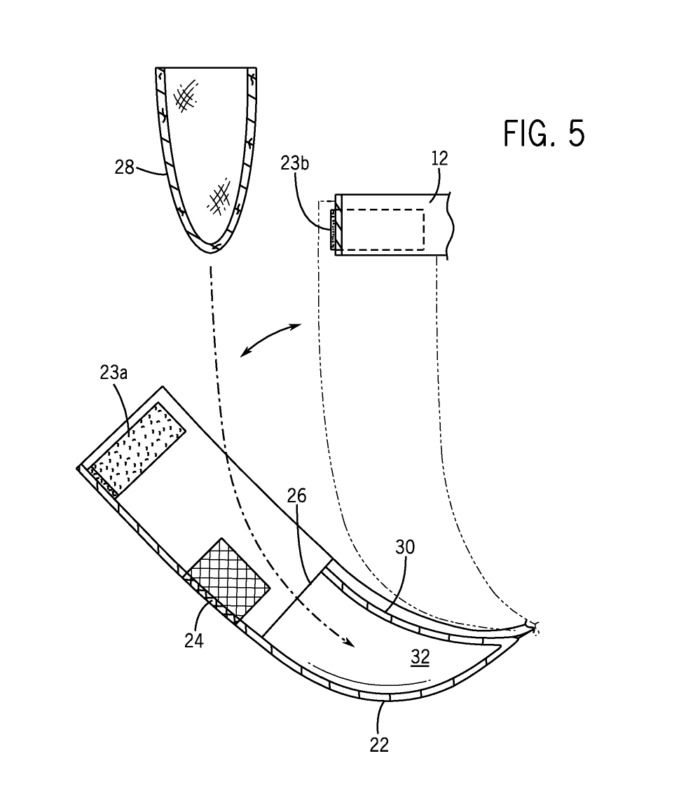

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

Referring now to the figures, the following reference numbers may refer to elements of the invention:
10: is the medical undergarment.
12: is the waistband.
14: is the user.
16: are the straps.
18: are the slits in waistband 12.
20: is the ‘S’ shaped hook length adjuster.
22: is the front panel.
23: is the fastener, such as (but not limited to) a hook and loop fastener.
23a: is the hook component of fastener 23.
23b: is the loop component of fastener 23.
24: is the mesh.
26: is the inner opening.
28: is the absorbent cup or insert.
30: is the inner panel.
32: is the cavity to receive insert 28.

An undergarment according to one embodiment of the present invention eliminates essentially all wetness to the outer clothing and the discomfort to the skin. The undergarment according to one embodiment of the present invention may include adjustable straps, making it invention custom fitting, and includes features to allow for easy and quick replacement of each pad.

The undergarment according to one embodiment of the present invention may take the shape of a jock strap with a pouch that holds absolvent material that enclose the male's penis and scrotum with adjustable straps and at least one fastener (such as Velcro™). The undergarment according to one embodiment of the present invention allows for replacing only the tube shaped pad.

In one aspect of the present invention, the elastic waistband 12 holds the apparatus onto the person's waist. It holds the front panel 22, that holds the absorbent pad or insert 28, in which the penis and scrotum sits, and two to four elastic cloth straps 16 adjust for custom fit.

Referring now to the drawings, in one aspect of the present invention, a medical undergarment 10 comprises: a waistband 12 configured to be worn on a waist of a man or user 14; a front panel 22 connectable to a front region (such as toward a front of the body of the user 14) of the waistband 12 and comprising an inner opening 26; an adjustable strap 16 connecting the front panel 22 to a rear region (such as toward a back of the body of the user 14) of the waistband 12; an absorbent cup 28 comprising an absorbent material, receivable in the inner opening 26 of the front panel 22, and configured to contain and absorb urine; and a fastener 23 configured to removably connect the front panel 22 to the front region of the waistband 12.

The waistband 12 may comprise any material, such as fabric, plastic, other textiles, elastic materials, etc., and may be adjustable in length to fit the waist of many or all men. The front panel 22 may comprise any material, such as fabric, plastic, other textiles, elastic materials, etc., and the inner opening 26 may comprise a pouch or holding device. It may have a similar or matching shape to absorbent cup 28, which may have a cup shape, approximately semi-ellipsoid shape (e.g., a bullet shape), or any shape to hold or contain a fluid, and may comprise any known absorbent material, including but not limited to cotton or other natural or synthetic fibers. The fastener 23 may include any fastener known to connect the front panel 22 to the waistband 12, such as snaps, adhesives, hook-and-loop (e.g., Velcro™) fasteners, hooks, buttons, and so forth.

In one aspect, the front panel 22 further comprises an adjustment device configured to hold at least one of a penis and a scrotum in place. In one aspect, the adjustment device comprises straps and/or an elastic band that may or may not run a periphery or circumference of an opening of the inner opening 26. In one aspect, the adjustment device comprises a fabric mesh 24. The adjustment device may encircle the penis, scrotum, or both, and may help to hold the penis in place inside the absorbent cup 28 and/or the inner opening 26. The adjustment device may also tightly wrap around the penis and/or scrotum so that liquid cannot easily escape or leak from the inner opening 26.

In one aspect, the medical undergarment further comprises a length adjustor, such as an ‘S’ shaped hook length adjustor 20, connected to the adjustable strap 16 and configured to allow the adjustable strap 16 to be adjusted in length. Any known length adjustor is within the scope of the present invention.

In one aspect, a method of collecting urine comprises: providing the medical undergarment 10 as described; inserting the absorbent cup 28 into the inner opening 26; placing a penis of a user into the absorbent cup 28; adjusting the adjustable strap 16; and connecting the front panel 22 to the front region of the waistband 12 with the fastener 23.

In one aspect, the front panel 22 further comprises an adjustment device, such as mesh 24, configured to hold the penis in place, and wherein the method further comprises adjusting the adjusting device so as to hold the penis in place. In one aspect, the adjustment device wraps tightly around the penis so as to prevent outflow of urine from the inner opening 26.

It should be understood, of course, that the foregoing relates to exemplary embodiments of the invention and that modifications may be made without departing from the spirit and scope of the invention.

What is claimed is:

1. A medical undergarment comprising:
a waistband configured to be worn on a waist of a man;
a front panel connectable to a front region of the waistband and comprising an inner opening leading into a pouch formed within the front panel;
an adjustable strap connecting the front panel to a rear region of the waistband;
an absorbent cup comprising a similar shape of the pouch and comprising an absorbent material, wherein the absorbent cup is separate from and receivable within the pouch, and configured to contain and absorb urine; and
a fastener configured to removably connect the front panel to the front region of the waistband.

2. The medical undergarment as claimed in claim 1, wherein the front panel further comprises an adjustment device configured to hold at least one of a penis and a scrotum in place.

3. The medical undergarment as claimed in claim 2, wherein the adjustment device comprises an elastic band.

4. The medical undergarment as claimed in claim 2, wherein the adjustment device comprises a fabric mesh.

5. The medical undergarment as claimed in claim 1, wherein the fastener comprises a hook-and-loop fastener.

6. The medical undergarment as claimed in claim 1, further comprising a length adjustor connected to the adjustable strap and configured to allow the adjustable strap to be adjusted in length.

7. The medical undergarment as claimed in claim 1, wherein the absorbent pad has an approximately semi-ellipsoid shape.

8. A method of collecting urine, comprising:
providing the medical undergarment as claimed in claim 1;
inserting the absorbent cup into the pouch through the inner opening;
placing a penis of a user into the absorbent cup;
adjusting the adjustable strap; and
connecting the front panel to the front region of the waistband with the fastener.

9. The method as claimed in claim 8, wherein the front panel further comprises an adjustment device configured to hold the penis in place, and wherein the method further comprises adjusting the adjusting device so as to hold the penis in place.

10. The method as claimed in claim 9, wherein the adjustment device wraps tightly around the penis so as to prevent outflow of urine from the inner opening.
